(19) United States
(12) Patent Application Publication
(10) Pub. No.: US 2020/0000633 A1
(43) Pub. Date: Jan. 2, 2020

(54) CRYOSPLINT

(71) Applicant: Sydney Taylor, Saratoga Springs, UT (US)

(72) Inventors: Sydney Taylor, Saratoga Springs, UT (US); Britnee Nicole Hogue, Saratoga Springs, UT (US); Trever Alan Whiting, West Jordan, UT (US)

(21) Appl. No.: 16/455,277

(22) Filed: Jun. 27, 2019

Related U.S. Application Data
(60) Provisional application No. 62/692,653, filed on Jun. 29, 2018.

(51) Int. Cl. A61F 7/02; A61F 5/01; A61F 7/10

(52) U.S. Cl. CPC A61F 7/02; A61F 5/0104; A61F 2007/0039; A61F 2007/0239; A61F 2007/0231; A61F 7/106

(57) ABSTRACT

Cryosplint. In some embodiments, a cryosplint may include a vacuum splint and a cold pack. The vacuum splint may be configured to be wrapped around a body part of a patient and then have air evacuated therefrom in order to stabilize the body part. The cold pack may be configured to be attached to the vacuum splint in order to provide application of cold to the body part simultaneous to the vacuum splint stabilizing the body part.

### CROSS-REFERENCE TO A RELATED APPLICATION

This application claims the benefit of, and priority to, U.S. Provisional Application No. 62/692,653, filed Jun. 29, 2018, which is incorporated herein by reference in its entirety.

### BACKGROUND

Conventional vacuum splints may be used to stabilize an injured body part of a patient, such as a fractured bone in an extremity. One advantage of a vacuum splint it that it may allow for effective emergency splinting of an injured body part while being radiolucent, in order to allow radiographic imaging of the body part to occur without creating any obstruction.

However, conventional vacuum splints can also impede or prevent the application of ice or other cold treatment to assist in the healing process and to reduce swelling to the injured body part. Without this reduction in swelling, radiographic imaging of the body part may be impaired.

The subject matter claimed herein is not limited to embodiments that solve any disadvantages or that operate only in environments such as those described above. Rather, this background is only provided to illustrate one example technology area where some embodiments described herein may be practiced.

### BRIEF DESCRIPTION OF THE DRAWINGS

Embodiments will be described and explained with additional specificity and detail through the use of the accompanying drawings in which:

FIGS. 1A-1B depict an example cryosplint secured to a leg of a patient;

FIG. 2A depicts an exterior of the example cryosplint of FIGS. 1A-1B;

FIG. 2B depicts an interior of the example cryosplint of FIGS. 1A-1B including a vacuum splint and cold packs attached thereto;

FIG. 2C depicts a side of the example cryosplint of FIGS. 1A-1B;

FIG. 2D depicts an interior of the example cryosplint of FIGS. 1A-1B with one of the cold packs of FIG. 2B being detached from the vacuum splint of FIG. 2B;

FIG. 3 depicts the cold packs of FIG. 2B detached from the vacuum splint of FIG. 2B; and

FIGS. 4A-4B depict charts showing the temperature of example cold packs over time.

### DETAILED DESCRIPTION

Conventional vacuum splints stabilize an injured body part of a patient while being radiolucent to allow radiographic imaging of the body part to occur without creating any obstruction. However, conventional vacuum splints can also impede or prevent the application of ice or other cold treatment to the injured body part to assist in the healing process and to reduce swelling. Without this reduction in swelling, radiographic imaging of the body part may be impaired.

The cryosplint disclosed herein may provide various benefits. In particular, the cryosplint disclosed herein may, for example, enable an injured body part of a patient to be stabilized while a cold treatment is simultaneously applied to the injured body part, for example to facilitate the patient being transported to a medical care facility where they can receive more advanced care the the injured body part being stabilized to decrease pain and prevent further injury. Further, the cryosplint disclosed herein may be radiolucent to allow radiographic imaging of the injured body part during the simultaneous stabilization and cold treatment, which may reduce swelling and thus enhance the radiographic imaging of the injured body part, for example to avoid the cryosplint from having to be removed prior to x-rays or MRI scans being taken of the injured body part. The simultaneous stabilization and cold treatment may also assist in the healing process of an injured body part.

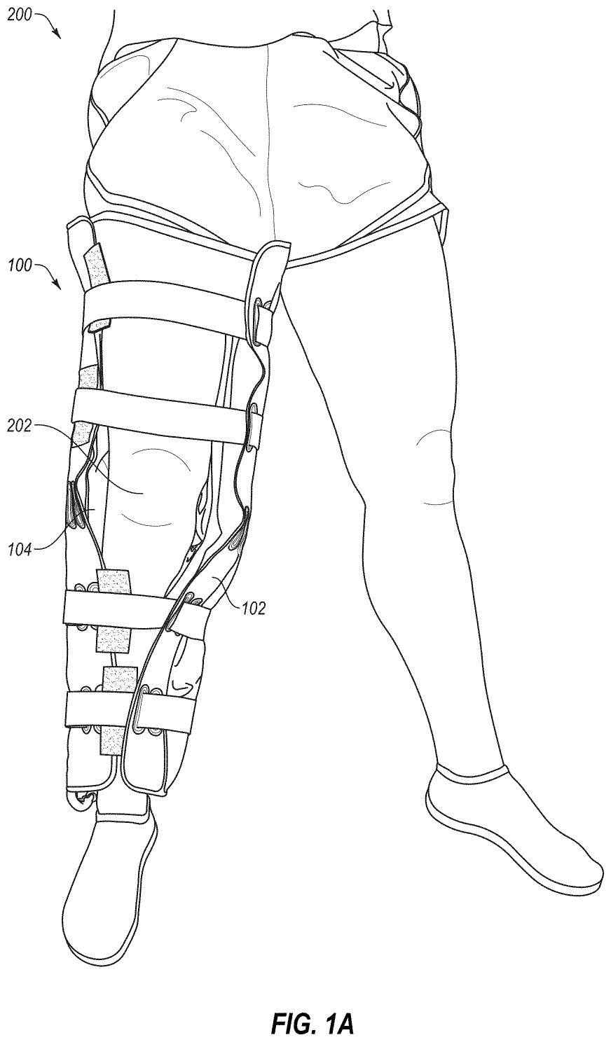

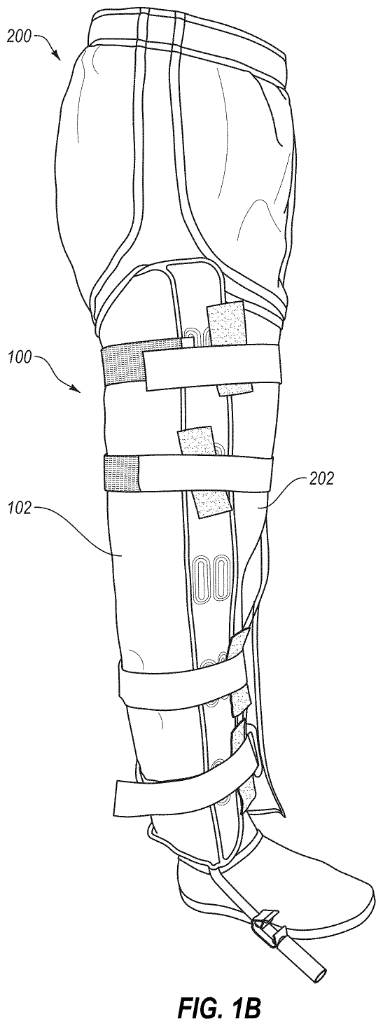

Turning to the figures, FIGS. 1A-1B depict an example cryosplint 100 secured to a leg 202 of a patient 200. As disclosed in FIGS. 1A-1B, the example cryosplint 100 may include a vacuum splint 102 and one or more cold packs 104. The vacuum splint 102 may be configured to be wrapped around the leg 202 or other body part of the patient 200 and then have air evacuated therefrom in order to stabilize the leg 202 or other body part. The cold packs 104 may be configured to be attached to the vacuum splint 102 in order to provide application of cold to the leg 202 or other body part simultaneous to the vacuum splint 102 stabilizing the leg 202 or other body part.

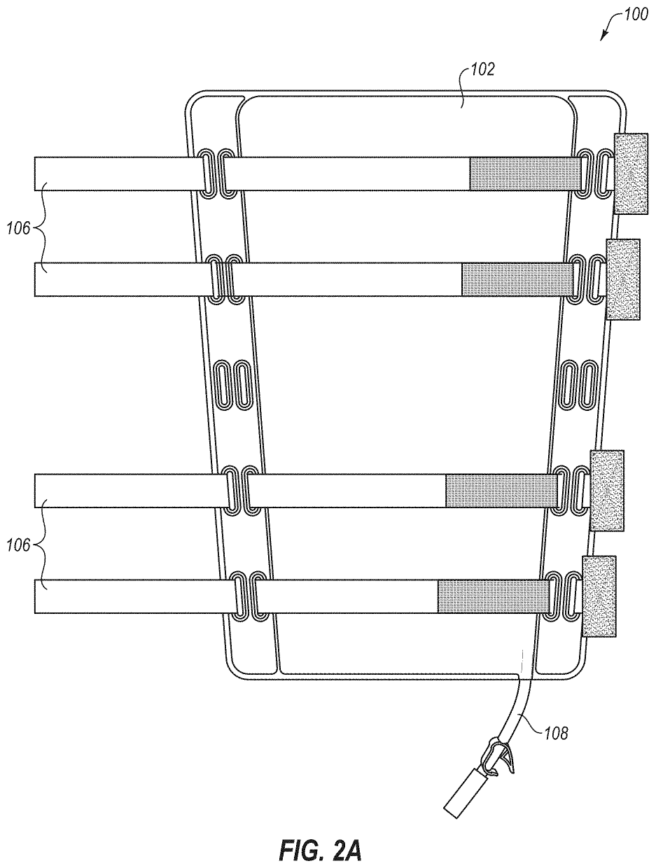

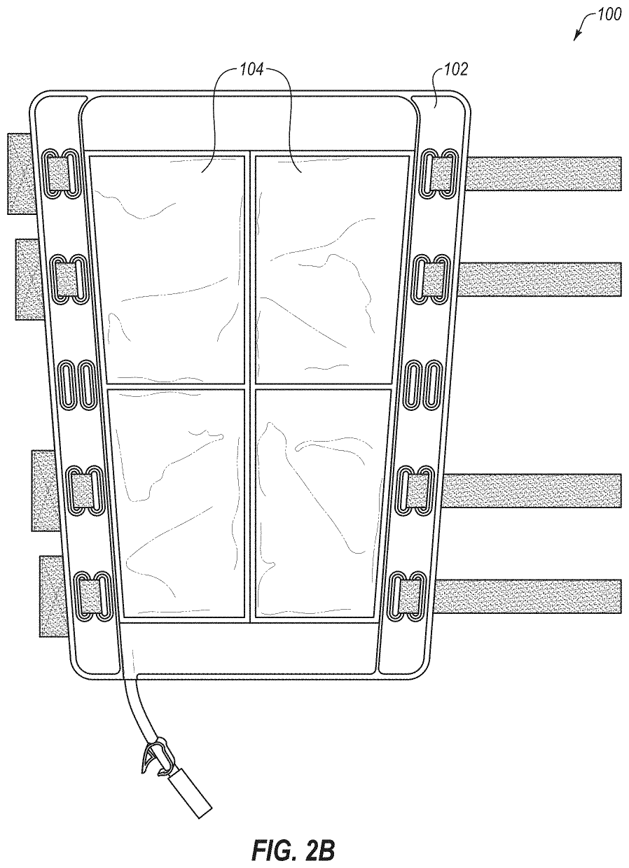

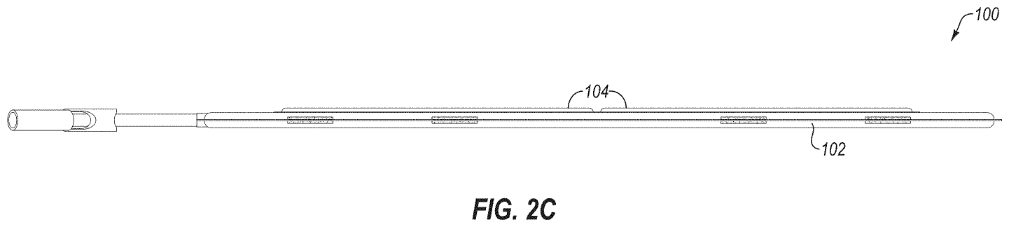

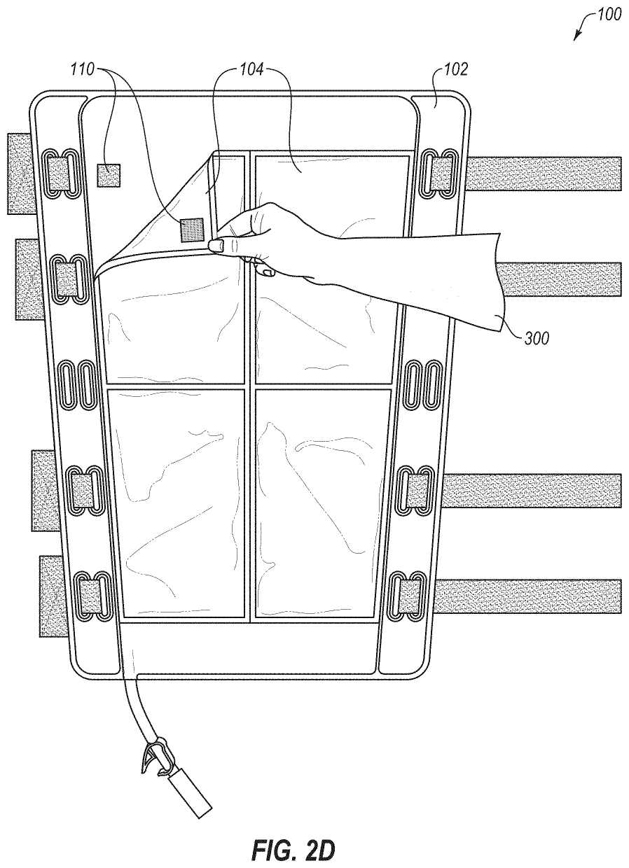

FIG. 2A depicts an exterior of the example cryosplint 100. As disclosed in FIG. 2A, the vacuum splint 102 of the cryosplint 100 may include straps 106 and a hose 108. After the cryosplint 100 is wrapped around a body part of a patient, the straps 106 may be fastened using fasteners, such as hook-and-loop fasteners, in order to secure the cryosplint 100 to the body part. Then, air may be evacuated from the vacuum splint 102 through the hose 108 (e.g. using a hand pump) in order to stabilize the body part.

FIG. 2B depicts an interior of the example cryosplint 100 including the vacuum splint 102 and cold packs 104 attached thereto. As disclosed in FIG. 2B, the vacuum splint 102 may have one or more cold packs 104 attached thereto, such that the vacuum splint 102 is lined with the cold packs 104 being attached to an interior surface of the vacuum splint 102. After the cryosplint 100 is wrapped around a body part of a patient, the cold packs 104 may apply cold to the body part simultaneous to the vacuum splint 102 stabilizing the body part.

FIG. 2C depicts a side of the example cryosplint 100. As disclosed in FIG. 2C, the cold packs 104 may only marginally add to the thickness of the vacuum splint 102. Therefore, this marginal addition to the thickness of the vacuum splint 102 may allow the vacuum splint 102 to function normally notwithstanding the presence of the cold packs 104 attached thereto.

FIG. 2D depicts an interior of the example cryosplint 100 with one of the cold packs 104 being detached from the vacuum splint 102. As disclosed in FIG. 2D, the one or more cold packs 104 may be configured to be temporarily attached to the vacuum splint 102 using fasteners, such as hook-and-loop fasteners 110 that are attached to the vacuum splint 102 and to the cold pack 104. Further, as disclosed in FIG. 2D, the cold packs 104 may be transparent such that the hook-and-loop fasteners 110 that are attached to the vacuum splint may be visible event when facing away from a user 300, such as an emergency medical professional. For example, the cold packs 104 may be constructed from clear vinyl sheeting.

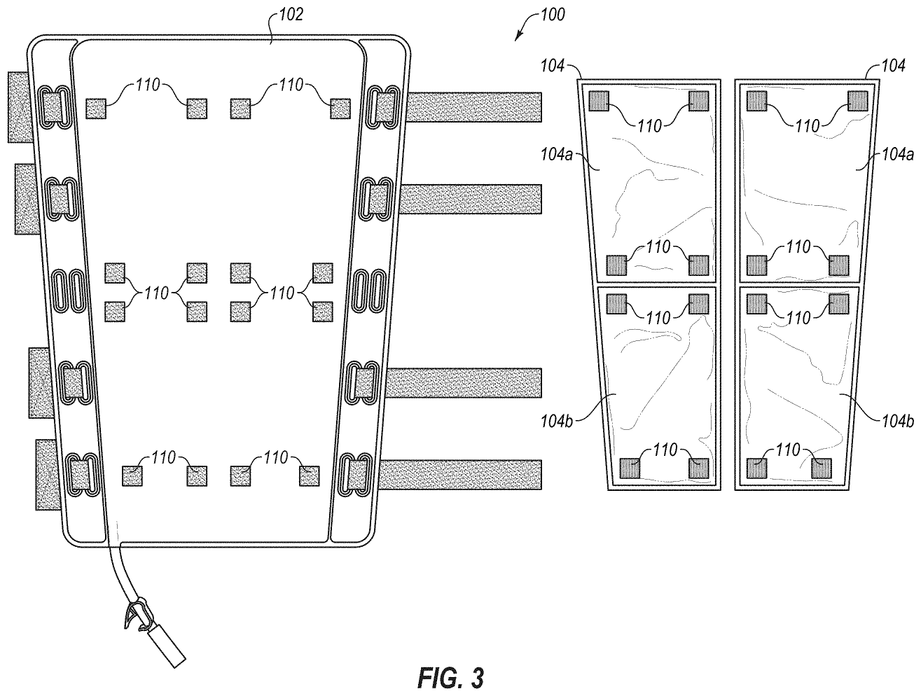

FIG. 3 depicts the cold packs 104 detached from the vacuum splint 102. As disclosed in FIG. 3, the hook-and-loop fasteners 110 that are attached to the vacuum splint 102 and to the cold pack 104 may be aligned such that the cold packs 104 can be easily attached to the vacuum splint 102. Further, as disclosed in FIG. 3, the loop (e.g., soft) portions of the hook-and-loop fasteners 110 may be attached to the vacuum splint 102, while the hook (e.g. scratchy) portions of the hook-and-loop fasteners 110 may be attached to the cold pack 104, so that in the event that the vacuum splint 102 is ever used without the cold pack 104, the loop (e.g., soft) portions of the hook-and-loop fasteners 110 are the portions that come in contact with the skin of a patient, which may cause less irritation than would be caused by the hook (e.g. scratchy) portions. Further, while the hook-and-loop fasteners 110 may be permanently attached to the vacuum splint 102 and to the cold pack 104, in some embodiments the hook-and-loop fasteners 110 may be removable (e.g. using a removable tape that can be peeled off), between uses of the cryosplint 100, so that they can be replaced with fresh, sterile replacements in order to make the vacuum splint 102, and/or the cold pack 104, sterile if reused.

In some embodiments, the cold packs 104 may be configured to activate to become colder without refrigeration. In these embodiments, the cold packs 104 may be activated by squeezing and/or shaking, which may result the rupture of internal packages, such as 2-mm expanded polystyrene ball, which may result in a chemical reaction which causes the cold packs 104 to transition from the temperature of the ambient air around the cold packs 104 to a colder temperature suitable for application against the skin of a patient, such as to reduce swelling after an injury. In these embodiments, the cold packs 104 may be single-use cold packs (e.g., disposable), while the vacuum splint 102 may be a reusable vacuum splint configured to be have old single-use cold packs 104 discarded and new single-use cold packs 104 attached thereto prior to each use. In other embodiments, the cold packs 104 may be reusable cold packs, such as conventional ice packs.

In some embodiments, the cold packs 104 may include multiple sealed chambers. For example, FIG. 3 discloses two cold packs 104, each with two chambers that include upper chambers 104a and lower chambers 104b. Having multiple chambers in a single cold pack 104 may prevent a liquid coolant within the cold pack from flowing entirely to one end of the cold pack. For example, when the cryosplint 100 is used in the manner disclosed in FIGS. 1A-1B on the leg 202 of a patient 200, having upper chambers 104a in each of the cold packs 104 may prevent all of the liquid coolant within the cold packs 104 from flowing to the bottom of the cold packs 104 when the patient 200 is standing while wearing the cryosplint 100. Instead, half of the liquid coolant in each of the cold packs 104 is forced to remain in the top half (e.g., in the upper chamber 104a) of each of the cold packs 104.

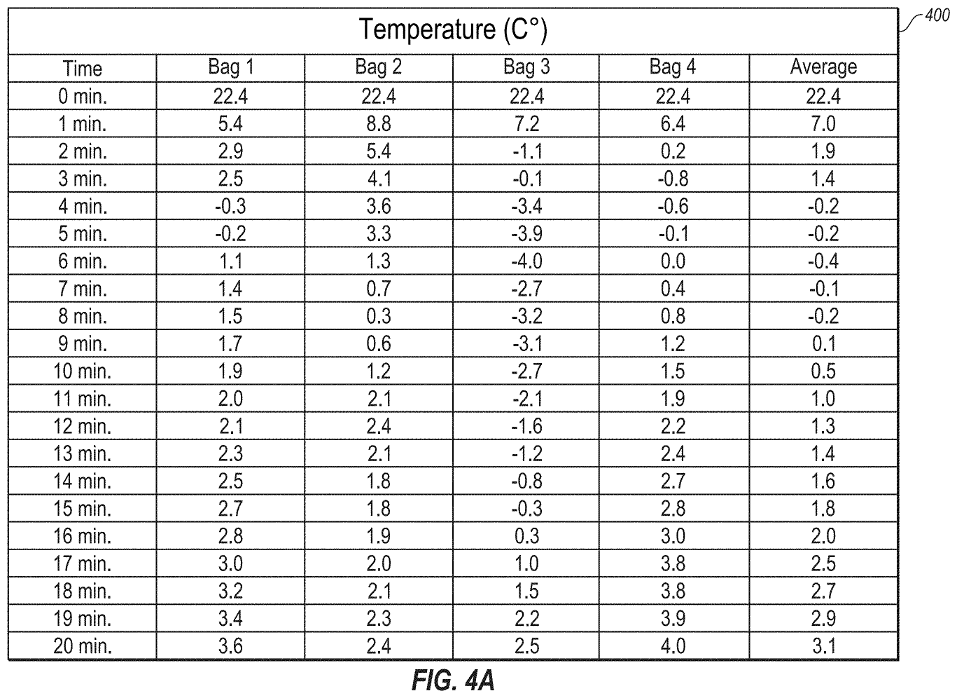

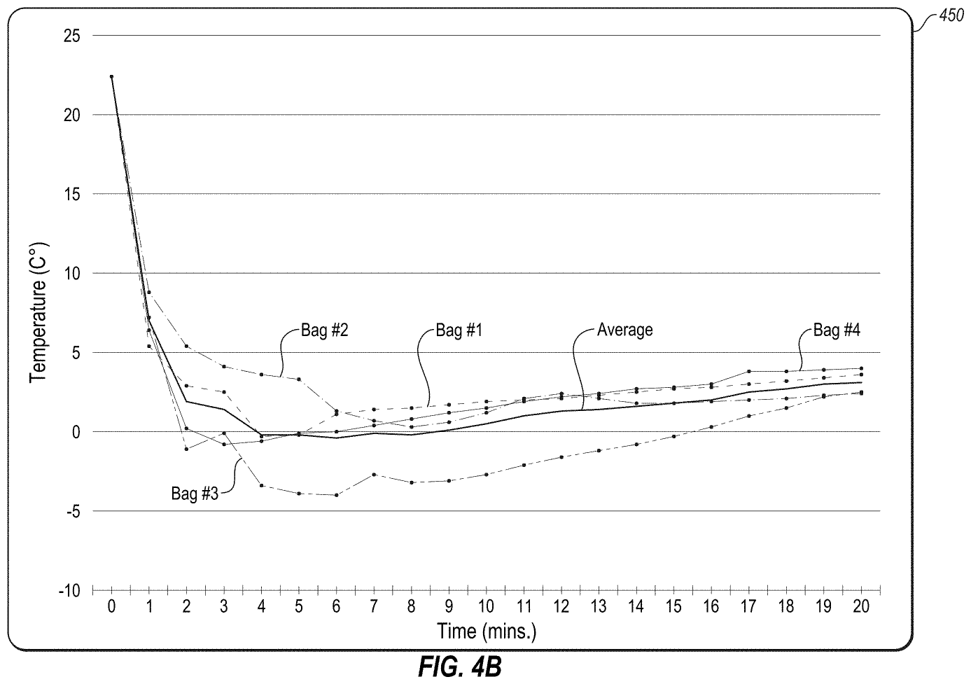

FIGS. 4A-4B depict charts 400 and 450 showing the temperature of example cold packs 104 over time. As disclosed in the charts 400 and 450, the average temperature of the cold packs between about 2 and 20 minutes after activation may allow the cold packs 104 to be acceptable for application against the skin of a patient, such as to reduce swelling after an injury. For example, the cold temperature of the cold packs 104 after activation may enable the cold packs 104 to cause veins under the skin to constrict to bring down swelling in the injured body part.

Although the example cryosplint 100 discussed above generally includes the vacuum splint 102 and one or more cold packs 104 attached to an interior surface of the vacuum splint 102, it is understood that other embodiments of a cryosplint may include a vacuum splint and one or more cold packs integrated into the vacuum splint. In these embodiments, the entire cryosplint may be single-use (e.g., disposable). These embodiments may be useful in emergency situations, such as in military or search-and-rescue situations.

In accordance with common practice, the various features illustrated in the drawings may not be drawn to scale. The illustrations presented in the present disclosure are not meant to be actual views of any particular apparatus (e.g., device, system, etc.) or method, but are merely example representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the summary, detailed description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

The foregoing description, for purpose of explanation, has been described with reference to specific embodiments. However, the illustrative discussions above are not intended to be exhaustive or to limit the invention as claimed to the precise forms disclosed. Many modifications and variations are possible in view of the above teachings. The embodiments were chosen and described to explain practical applications, to thereby enable others skilled in the art to utilize the invention as claimed and various embodiments with various modifications as may be suited to the particular use contemplated.

1. A cryosplint comprising:
a vacuum splint configured to be wrapped around a body part of a patient and then have air evacuated therefrom in order to stabilize the body part; and
a cold pack configured to be attached to the vacuum splint in order to provide application of cold to the body part simultaneous to the vacuum splint stabilizing the body part.

2. The cryosplint of claim 1, wherein the cold pack is configured to be temporarily attached to the vacuum splint using hook-and-loop fasteners that are attached to the vacuum splint and to the cold pack.

3. The cryosplint of claim 2, wherein the cold pack is transparent such that the hook-and-loop fasteners that are attached to the vacuum splint are visible event when facing away from a user.

4. The cryosplint of claim 1, wherein the cold pack is a single-use cold pack.

5. The cryosplint of claim 4, wherein the single-use cold pack is configured to activate to become colder without refrigeration.

6. The cryosplint of claim 4, wherein the vacuum splint is a reusable vacuum splint configured to be used repeatedly.

7. The cryosplint of claim 1, wherein the cold pack includes multiple sealed chambers.

8. The cryosplint of claim 7, further comprising a second cold pack configured to be attached to an interior surface of the vacuum splint and including multiple sealed chambers.

9. A cryosplint comprising:
a vacuum splint configured to be wrapped around a body part of a patient and then have air evacuated therefrom in order to stabilize the body part; and
a cold pack configured to be temporarily attached to the vacuum splint using hook-and-loop fasteners that are attached to the vacuum splint and to the cold pack in order to provide application of cold to the body part simultaneous to the vacuum splint stabilizing the body part.

10. The cryosplint of claim 9, wherein the cold pack is transparent such that the hook-and-loop fasteners that are attached to the vacuum splint are visible event when facing away from a user.

11. The cryosplint of claim 9, wherein the cold pack is a single-use cold pack.

12. The cryosplint of claim 11, wherein the cold pack is configured to activate to become colder without refrigeration.

13. The cryosplint of claim 11, wherein the vacuum splint is a reusable vacuum splint configured to be used repeatedly.

14. The cryosplint of claim 9, wherein the cold pack includes multiple sealed chambers.

15. The cryosplint of claim 14, further comprising a second cold pack configured to be attached to an interior surface of the vacuum splint and including multiple sealed chambers.

16. A cryosplint comprising:
a vacuum splint configured to be wrapped around a body part of a patient and then have air evacuated therefrom in order to stabilize the body part; and
a cold pack including multiple sealed chambers and configured to be attached to the vacuum splint in order to provide application of cold to the body part simultaneous to the vacuum splint stabilizing the body part.

17. The cryosplint of claim 16, wherein:
the cold pack is configured to be temporarily attached to the vacuum splint using hook-and-loop fasteners that are attached to the vacuum splint and to the cold pack; and
the cold pack is transparent such that the hook-and-loop fasteners that are attached to the vacuum splint are visible event when facing away from a user.

18. The cryosplint of claim 16, wherein:
the cold pack is a single-use cold pack; and
the cold pack is configured to activate to become colder without refrigeration.

19. The cryosplint of claim 18, wherein the vacuum splint is a reusable vacuum splint configured to be used repeatedly.

20. The cryosplint of claim 16, further comprising a second cold pack configured to be attached to the vacuum splint and including multiple sealed chambers each having a liquid coolant contained therein.
